(19) United States
(12) Patent Application Publication
(10) Pub. No.: US 2014/0257019 A1
(43) Pub. Date: Sep. 11, 2014

(54) Methods, Systems, and Devices Relating to a Fail-Safe Pump for a Medical Device

(71) Applicant: Sunshine Heart Company Pty, Ltd., New South Wales (AU)

(72) Inventors: Martin Cook, Eden Prairie, MN (US); Will Peters, Auckland (NZ); Dan Lafontaine, Plymouth, MN (US); Phillip J. Miller, Berkeley, CA (US); Steven Paul Woodard, Cupertino, CA (US); Gregory W. Hall, Los Gatos, CA (US)

(73) Assignee: Sunshine Heart Company Pty, Ltd., New South Wales (AU)

(21) Appl. No.: 14/197,365

(22) Filed: Mar. 5, 2014

Related U.S. Application Data
(60) Provisional application No. 61/772,707, filed on Mar. 5, 2013.

(51) Int. Cl. A61M 1/12; A61M 1/10

(52) U.S. Cl. CPC A61M 1/122; A61M 1/1072; A61M 1/1008; A61M 1/1043; A61M 1/1012
USPC 600/18; 600/16

(57) ABSTRACT

The various embodiments herein relate to pumps for use with various medical devices. The pumps can be positive displacement pumps or gear pumps. Each pump has at least one fluid transfer opening defined in the pump that allows for transfer of fluid at a predetermined flow rate that provides for deflation of the device in a predetermined amount of time.

### CROSS-REFERENCE TO RELATED APPLICATION(S)

This application claims priority to U.S. Provisional Application 61/772,707, filed on Mar. 5, 2013 and entitled “Methods, Systems, and Devices Relating to a Fail-Safe Pump for a Heart Assist Device,” which is hereby incorporated herein by reference in its entirety.

### FIELD OF THE INVENTION

The various embodiments disclosed herein relate to various fail-safe pumps for use in medical devices. More specifically, each pump has a fluid transfer opening that allows for some back flow (or “leakage”) of fluid in the event of an unexpected or unintended stoppage of the pump, thereby allowing for reduction of potentially damaging or dangerous pressure resulting from such stoppage.

### BACKGROUND OF THE INVENTION

Various heart assist devices can be used to treat end-stage heart failure, including, for example, left ventricular assist devices (“LVADs”), intra-aortic balloon devices, aortic compression devices, and other counterpulsation devices, among others.

Many of these assist devices are actuated by fluid pressure generated by a pump. In some cases, the pump is implanted inside the patient's body, while in other cases it is positioned outside the body. The pump provides fluid pressure to the device, thereby inflating the device, and then reduces the fluid pressure to the device, either actively or passively.

One risk of these pressure actuated systems relates to possible deflation failure. That is, if the pump or the entire system inadvertently or unexpectedly fails during the inflation cycle, the inflated device remains inflated, which can result in injury or even death for the patient or damage to the device.

There is a need in the art for an improved pump for use with heart assist devices.

### BRIEF SUMMARY OF THE INVENTION

Discussed herein are various systems and devices relating to displacement and gear pumps, each having at least one fluid transfer opening that allows some predetermined amount of fluid leakage to reduce fluid pressure in case of an unintentional or unexpected stoppage.

In Example 1, an pump for a medical device comprises a body defining an interior, a displacement component disposed within the interior, a first chamber, a second chamber, a conduit, and at least one fluid transfer opening. The first chamber is defined by a distal portion of the body and a distal side of the displacement component. The conduit is in fluid communication with the first chamber and further is in fluid communication with the medical device. The second chamber is defined by a proximal portion of the body and a proximal side of the displacement component. The at least one fluid transfer opening defined between the first chamber and the second chamber

Example 2 relates to the pump according to Example 1, wherein the medical device is an inflatable compression device.

Example 3 relates to the pump according to Example 2, wherein the at least one fluid transfer opening is sized and shaped to allow the compression device to deflate within a time period ranging from about 10 seconds to about 30 seconds.

Example 4 relates to the pump according to Example 2, wherein the at least one fluid transfer opening is sized and shaped to allow a maximum flow rate through the opening of about 2 cc per second.

Example 5 relates to the pump according to Example 1, wherein the displacement component comprises a displacement wall.

Example 6 relates to the pump according to Example 5, wherein the at least one fluid transfer opening comprises an opening defined in the displacement wall.

Example 7 relates to the pump according to Example 6, further comprising a non-rigid coupling component operably coupled to the displacement wall and an interior wall of the body.

Example 8 relates to the pump according to Example 5, wherein the at least one fluid transfer opening comprises a gap between the displacement wall and an interior wall of the body.

Example 9 relates to the pump according to Example 5, wherein the at least one fluid transfer opening comprises a bypass chamber defined in the body.

Example 10 relates to the pump according to Example 9, wherein the displacement wall is positioned adjacent to the bypass chamber when the displacement wall is in a deflation position.

Example 11 relates to the pump according to Example 5, wherein the at least one fluid transfer opening comprises at least one slot defined in the displacement wall, wherein the implantable pump further comprises at least one projection shaped to fit within the slot.

Example 12 relates to the pump according to Example 11, wherein the at least one projection is disposed within the at least one slot when the displacement wall is in an inflation position.

Example 13 relates to the pump according to Example 1, wherein the displacement component comprises an at least one rotor.

Example 14 relates to the pump according to Example 1, wherein the displacement component comprises a first rotor and a second rotor.

In Example 15, an pump for a medical device comprises a body defining an interior, a displacement wall disposed within the interior, a first chamber, a second chamber, a conduit, a compliance chamber, and at least one fluid transfer opening. The first chamber is defined by a distal portion of the body and a distal side of the displacement wall. The conduit is in fluid communication with the first chamber and in fluid communication with the medical device. The second chamber is defined by a proximal portion of the body and a proximal side of the displacement wall. The compliance chamber is in fluid communication with the second chamber. The at least one fluid transfer opening is defined between the first chamber and the second chamber.

Example 16 relates to the pump according to Example 15, wherein the at least one fluid transfer opening comprises an opening defined in the displacement wall.

Example 17 relates to the pump according to Example 15, wherein the at least one fluid transfer opening comprises a gap between the displacement wall and an interior wall of the body.

In Example 18, an gear pump for a medical device comprises a body defining an interior, at least one rotor disposed within the interior, a first chamber, a second chamber, a conduit, and at least one fluid transfer opening. The first chamber is defined by a distal portion of the body and a distal portion of the at least one rotor. The conduit is in fluid communication with the first chamber, the conduit being in fluid communication with the medical device. The second chamber is defined by a proximal portion of the body and a proximal portion of the at least one rotor. The at least one fluid transfer opening is defined between the first chamber and the second chamber.

Example 19 relates to the pump according to Example 18, wherein the at least one rotor comprises a first rotor and a second rotor.

Example 20 relates to the pump according to Example 18, wherein the at least one fluid transfer opening comprises a gap between the at least one rotor and an interior wall of the body.

While multiple embodiments are disclosed, still other embodiments of the present invention will become apparent to those skilled in the art from the following detailed description, which shows and describes illustrative embodiments of the invention. As will be realized, the invention is capable of modifications in various obvious aspects, all without departing from the spirit and scope of the present invention. Accordingly, the drawings and detailed description are to be regarded as illustrative in nature and not restrictive.

### BRIEF DESCRIPTION OF THE DRAWINGS

FIG. 1A is a perspective view of a heart assist device system, according to one embodiment.

FIG. 1B is a schematic view of the heart assist device system, according to the embodiment of FIG. 1A.

FIG. 2 is a cutaway cross-sectional view of a positive displacement pump, according to one embodiment.

FIG. 3 is a cutaway cross-sectional view of a positive displacement pump, according to another embodiment.

FIG. 4 is a perspective view of a known roller screw drive system.

FIG. 5 is a cutaway cross-sectional view of an internal gear pump, according to one embodiment.

FIG. 6 is a cutaway cross-sectional view of an internal gear pump, according to another embodiment.

FIG. 7 is a cutaway cross-sectional view of a known external gear pump.

FIG. 8 is a cutaway cross-sectional view of an external gear pump, according to one embodiment.

FIG. 9A is a cutaway cross-sectional view of a set of rotatable internal magnets of a motor assembly, according to one embodiment.

FIG. 9B is a cutaway cross-sectional view of the motor assembly according to the embodiment of FIG. 9A.

FIG. 10 is a cutaway cross-sectional side view of a positive displacement pump, according to one embodiment.

FIG. 11A is a cutaway cross-sectional exploded perspective view of a portion of a positive displacement pump, according to one embodiment.

FIG. 11B is another cutaway cross-sectional exploded perspective view of the positive displacement pump according to the embodiment of FIG. 11A.

FIG. 12A is a top view of a positive displacement pump, according to one embodiment.

FIG. 12B is a cutaway cross-sectional side view of the positive displacement pump according to the embodiment of FIG. 12A.

FIG. 12C is another cutaway cross-sectional side view of the positive displacement pump according to the embodiment of FIGS. 12A and 12B.

FIG. 13A is a cutaway cross-sectional top view of a positive displacement pump, according to one embodiment.

FIG. 13B is a cutaway cross-sectional side view of the positive displacement pump embodiment of FIG. 13A.

FIG. 14A is a cutaway cross-sectional side view of a positive displacement pump, according to another embodiment.

FIG. 14B is another cutaway cross-sectional side view of the positive displacement pump embodiment of FIG. 14A.

### DETAILED DESCRIPTION

The various embodiments disclosed herein relate to pumps for use in various medical device systems, including, for example, mechanical heart assist device systems.

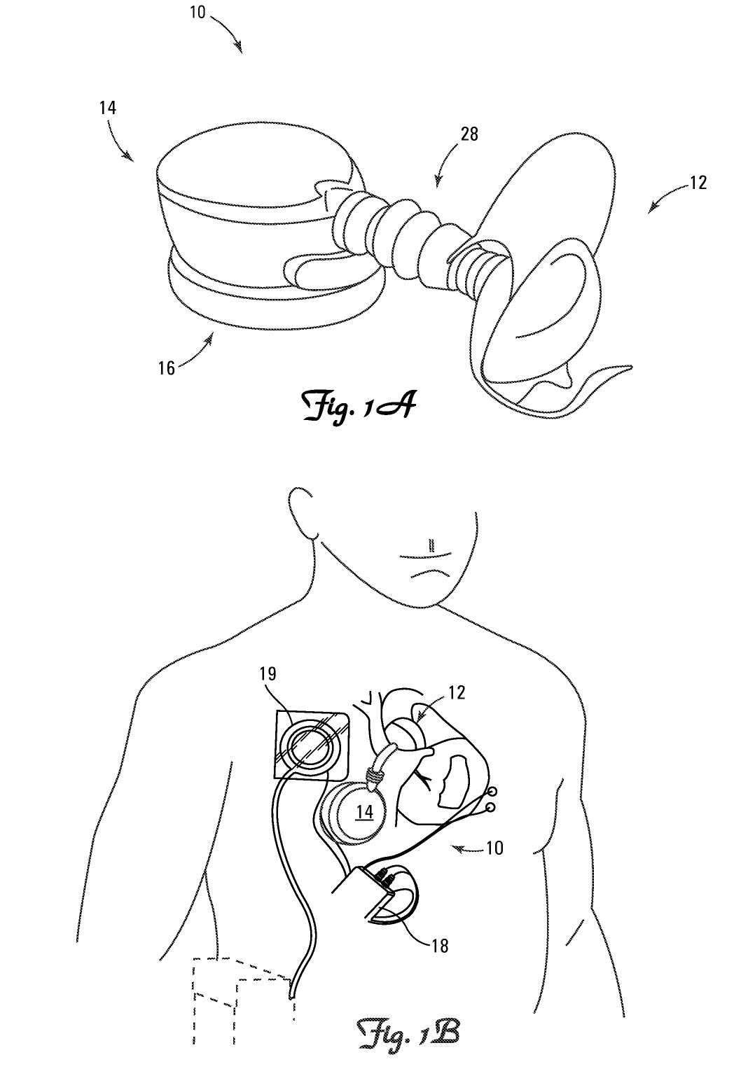

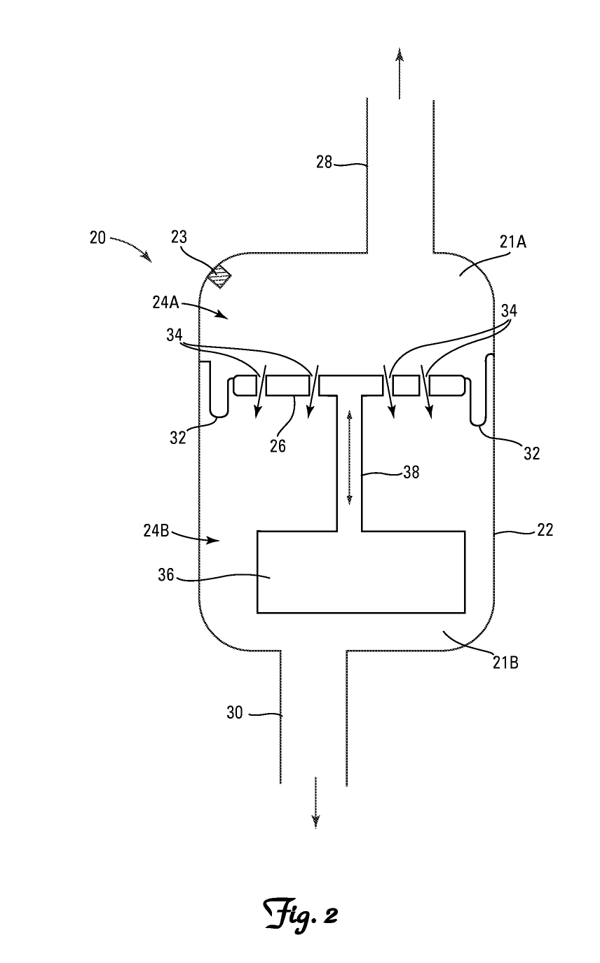

FIGS. 1A and 1B depict a heart assist device system 10, according to one embodiment. In this particular embodiment, the device 12 is an aortic compression device 12 that is configured to be positioned against the patient's ascending aorta and is configured to compress the ascending aorta and thereby assist in urging blood through the aorta and to the patient's body. The aortic compression device 12 is coupled to and in fluid communication—via a first fluidic coupling component 28—with a fluid pump 14, which is configured to transfer fluid in a repeating or cyclic fashion between the pump 14 and the compression device 12 via the coupling component 28, thereby providing the motive force that causes the device 12 to inflate and thereby compress the aorta and then either causes the device 12 to deflate or allows the device 12 to deflate via aortic pressure. The pump 14 is also coupled to and in fluid communication with a compliance chamber 16. The compliance chamber 16 is configured to allow for volume changes in the pump as a result of the action of the pump transferring fluid to and from the compression device 12. In accordance with one implementation, the compliance chamber 16 is in contact with the patient's lung, because, as is understood in the art, the volume of the lung can change easily and the volume of the chamber 16 is comparatively small in comparison to the lung volume, thereby providing a compliant region in the patient's body for the compliance chamber 16 to be positioned.

In certain implementations, the compliance chamber 16 is an integral part of the pump 14, as shown in FIG. 1A. That is, in this example, the chamber 16 is a flexible wall of the pump 14. Alternatively, the compliance chamber 16 can be a separate component in fluid communication with the pump 14. In a further embodiment, the compliance chamber can be any embodiment of compliance chamber as described in U.S. Pat. No. 7,306,558, which is hereby incorporated herein by reference in its entirety.

Alternatively, for any of the embodiments disclosed or contemplated herein, the system can have a compression device that is positioned against a blood sac, a heart ventricle, or any blood conduit (including any blood vessel or artery) of a patient and is configured to compress that sac, ventricle, or conduit and thereby assist in urging blood through the patient's body. According to certain implementations, the device is a counter-pulsation device. Alternatively, the device can be a co-pulsation device.

Various embodiments disclosed herein relate to pumps, any of which can be used as the pump 14 in the system 10 of FIGS. 1A and 1B. It is understood that the term “pump” as used herein is not intended to be limiting, but is intended to mean any device or component that can generate fluid pressure and thereby actuate the compression device to cyclically or repeatedly compress a blood sac, a heart ventricle, any blood conduit, or the aorta of the patient. It is further understood that the pump can further be any pump that is configured to be coupled to a medical device for purposes of actuating the device in some way, including any implantable pump or any pump that is not implanted in the patient's body.

FIG. 2 depicts a pump 20, according to one embodiment. The pump 20 is a positive displacement pump 20 in which a component 26 in contact with the fluid 21 (which is identified as fluid 21A and fluid 21B as described below in further detail) is displaced through a known and controlled distance and thus displaces a known and controlled volume of the fluid 21. More specifically, the pump 20 in FIG. 2 is a dual chamber pump 20 having a pump body 22 that contains two chambers 24A, 24B. The two chambers 24A, 24B are separated by a moveable wall 26. For purposes of this application, “moveable wall” means any surface, wall, or component that separates the two chambers and can move between two positions within the pump body 22: an inflation position (in which the compression device 12 is inflated) and a deflation position (in which the compression device 12 is deflated). In the specific embodiment depicted in FIG. 2, the moveable wall 26 moves laterally between two positions in the body 22 as described in further detail below. The first chamber 24A contains a first volume of fluid 21A and is in fluid communication with the compression device 12 via a first fluidic coupling component 28. The second chamber 24B contains a second volume of fluid 21B and is in fluid communication with a compliance chamber—such as the compliance chamber 16 of FIG. 1A—via a second fluidic coupling component 30.

According to any of the embodiments disclosed or contemplated herein, the body (such as body 22) can be made of any biocompatible metal, polymeric material, or ceramic material. In certain specific implementations, the body can be made of a specific biocompatible metal such as a titanium alloy (such as Ti6Al4V), a commercially-available pure titanium, or a similar metal. Alternatively, the body can be made of a specific polymeric material such as polyether ether ketone (“PEEK”), Torlon® polyamide-imide (“PAI”), or a similar polymeric material. In a further alternative, the body can be made of Bionate®.

The moveable wall in any of the implementations herein (including, for example, wall 26) can be made of any known material for use in a medical device, including materials that are not biocompatible. In certain exemplary embodiments, the wall can be made of the same material(s) as the body as described above. In one example, the wall can be made of stainless steel or any other similar metal. Alternatively, the wall can be made of non-biocompatible metals. In a further alternative, the wall can be treated or coated to increase wear resistance. For example, the wall can be treated with a treatment such as nitriding the surface or any other known treatment for medical device components to increase wear resistance. In other examples, the wall can be coated with a coating such as a diamond-lie-carbon coating or any other known coating for medical device components to increase wear resistance.

Alternatively, the compliance chamber is an integral part of the pump 20 (such as a flexible wall) as described above. In such an embodiment, there is no second fluidic coupling component 30.

In a further alternative, the first fluidic coupling component 28 is configured to have compliant walls. That is, the walls of the component 28 are made of a flexible, elastic, or otherwise compliant material that allows the walls to be compliant in circumstances that the first chamber 24A exceeds a predetermined level of pressure that could potentially be damaging to the pump 20 or the medical device coupled to the coupling component 28.

The moveable wall 26 separates the first and second fluids 21A, 21B in the first and second chambers 24A, 24B, respectively. To maintain the desired separation, the wall 26 has a non-rigid coupling component 32 attached at each end of the wall 26, wherein each such coupling component 32 is attached at its other end to the inner wall of the pump body 22. As such, the non-rigid coupling components 32 make it possible for the wall 26 to move laterally within the pump body 22 while maintaining a fluidic seal between the moveable wall 26 and the inner walls of the pump body 22. The first fluid 21A is urged between the pump 14 and the compression device 12. The second fluid 21B is urged between the pump 14 and a compliance chamber 16. According to one embodiment, the first and second fluids 21A, 21B can be the same fluid or type of fluid.

In accordance with one implementation, the non-rigid coupling component (such as non-rigid coupling component 32) is a flexible component. In the implementation shown in FIG. 2, the component 20 is a known rolling diaphragm configuration and is made of a woven fabric impregnated with an elastomer, or a similar material. Alternatively, the non-rigid coupling component 32 is made of Biospan® segment polyurethane, or a similar material. Alternatively, the component 32 is elastic. In a further implementation, the component 32 is any known flexible material that has a high flex life.

It is understood that the fluid or fluids (such as fluids 21A, 21B) used in any positive displacement pump disclosed or contemplated herein can be any known liquid or gas for use in a medical device that utilizes fluid compressive force. In one implementation, the fluid 21 is silicone oil. One specific silicone oil example is Nusil® MED-368. Alternatively, the fluid 21 is saline. In a further alternative, the fluid 21 consists of any known fluid that provides good tribological properties, is hydrophobic, or is biocompatible. In a further implementation, the fluid has a viscosity in the range of from about 5 mPa·s to about 60 mPa·s. In a further embodiment, the fluid 21 is any biocompatible and sterilizable fluid that can be used in medical devices implanted inside the human body.

In the embodiment depicted in FIG. 2, the moveable wall 26 does not provide a complete fluidic seal between the first and second chambers 24A, 24B. Instead, the wall 26 has one or more fluid transfer holes, gaps, or openings 34 defined in the wall 26 that allow some amount of fluid 21 to travel from one of the chambers 24A, 24B to the other through the one or more openings 34. It is understood that for purposes of this application, the terms “fluid transfer hole” and “fluid transfer opening” are intended to mean any opening of any kind or shape defined in the moveable wall 26 or elsewhere between the first and second chambers 24A, 24B that is configured to allow for the transfer of fluid between the two chambers 24A, 24B. In the depicted implementation, the moveable wall 26 has four fluid transfer holes 34. Alternatively, the wall 26 can have a number of fluid transfer holes ranging from one hole to any number of holes that allows the appropriate amount of fluid 21 to flow at a desired rate from one chamber to the other. According to one embodiment, the fluid 21 flows from the chamber under higher pressure to the chamber of lower pressure.

In accordance with one implementation, the one or more fluid transfer holes 34 in the moveable wall 26 are configured to allow the compression device 12 to deflate over a relatively short period of time in the event of an unexpected or unintended stoppage of the pump 14. That is, if the pump 14 stops operating unexpectedly in a position such that, for example, the moveable wall 26 is positioned at or near the inflation position such that the compression device 12 is inflated (or in any state of inflation from partially inflated to fully inflated) and thus compressing the aorta, a predetermined flow or leakage rate of fluid 21 from the first chamber 24A to the second chamber 24B reduces the pressure in the first chamber 24A by a predetermined amount. The predetermined reduction of pressure in the first chamber 24A causes the deflation of the compression device 12 at a predetermined minimum rate despite the failure of the moveable wall 26 to move back toward the deflation position, thereby preventing any long term partial occlusion of the aorta and thus preventing any adverse effect on the patient as a result of the pump stoppage. Similarly, any compression device for use with any blood sac, a heart ventricle, or any blood conduit as described above would also benefit from this predetermined flow or leakage rate, thereby preventing any long term partial occlusion of any such sac, ventricle, or conduit and thus preventing any adverse effect on the patient.

In one embodiment, the one or more fluid transfer holes 34 cause the compression device 12 to substantially deflate within about 30 seconds in the case of a pump stoppage. Alternatively, the compression device 12 substantially deflates within a time ranging from about 10 seconds to about 30 seconds. In a further alternative, the device 12 substantially deflates within about 15, 20, or 25 seconds, or any range therein. In a further embodiment, the device 12 substantially deflates at a maximum rate of about 2 cc per second. It is understood that, in certain implementations, the deflation rates disclosed here apply to the gear pump embodiments discussed below.

Of course, the presence of the one or more fluid transfer holes 34 in the moveable wall 26 causes some leakage of fluid 21 from one chamber to the other during normal use of the pump 20, thereby causing the inflated compression device 12 to deflate slightly. If the deflation amount were to be unchecked during normal use, it is possible that at some amount of deflation beyond a certain level, the inflated compression device 12 would no longer compress the sac, ventricle, or conduit sufficiently to assist in urging blood through the patient's body or such assistance would be minimal and thus ineffective. Thus, in certain implementations, the number and size of the fluid transfer holes 34 are predetermined based on the size of the pump, the amount of fluid 21 in the system 10, and certain other parameters to ensure that the deflation during normal operation is negligible or minimal (not impacting the normal compression action of the compression device 12) while ensuring deflation of the device 12 within a desired amount of time in the event of a stoppage of the pump 20. This minimization of the deflation rate during normal use explains the maximum deflation rate of about 2 cc per second in certain embodiments as described above. Alternatively, the maximum deflation rate can be any rate at which the compression device 12 can still effectively compress the sac, ventricle, or conduit but beyond which the leakage causes the device 12 to be unable to compress the sac, ventricle, or conduit sufficiently to assist in urging blood through the patient's body.

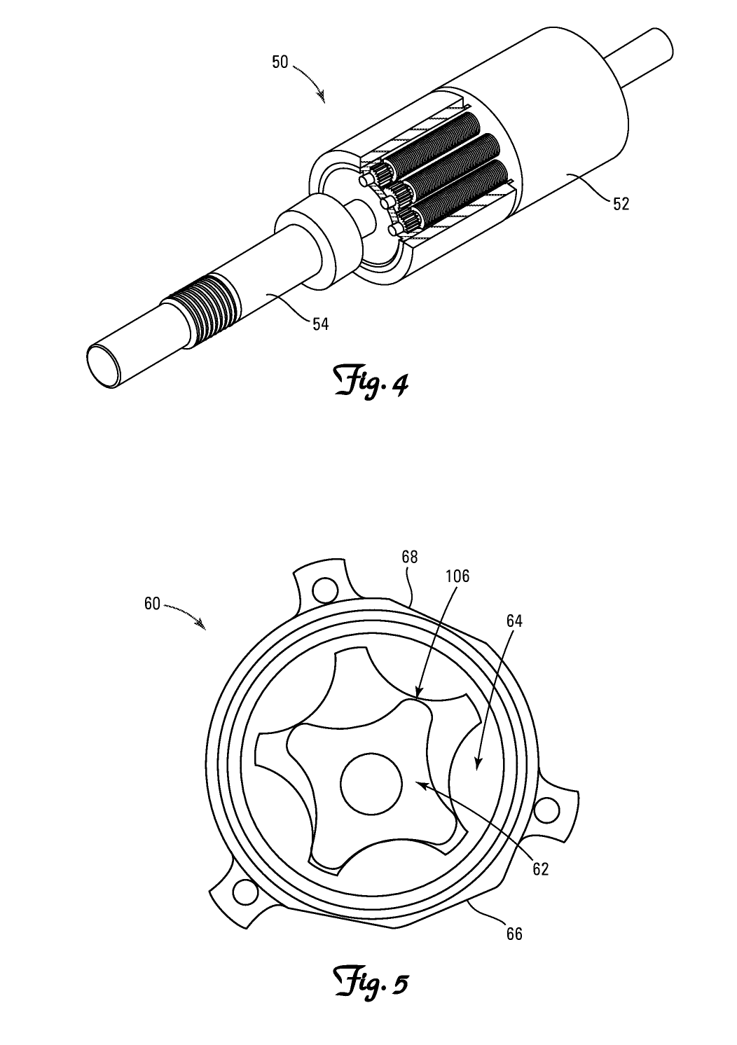

In one embodiment, the moveable wall 26 in the pump 20 (or any other positive displacement pump embodiment) is moved back and forth laterally using a motor 36 that is coupled to the wall 26 via an actuation arm 38. In one specific implementation, the moveable wall 26 is actuated using a known roller screw drive system 50 as shown in FIG. 4. The system 50 has a rotating drive component 52 that is coupled to the drive arm 54 such that the rotation of the component 52 causes the drive arm 54 to move laterally. That is, a motor (not shown) coupled with the rotating drive component 52 causes the drive component 52 to rotate. The drive component 52 is coupled to the drive arm 54 such that rotation of the component 52 causes the arm 54 to move laterally along the longitudinal axis of the system 50. The arm 54 is coupled with the moveable wall 26 such that that the lateral movement of the arm 54 causes lateral movement of the wall 26 toward and away from the motor 52.

Alternatively, a ball screw drive system could be used with any positive displacement pump implementation. In a further alternative, any known motor for use in medical devices that can actuate the wall 26 to move laterally can be used in any positive displacement pump contemplated herein.

Returning to FIG. 2, in accordance with one implementation, a pressure sensor 23 is provided in the pump body 22 that senses fluid pressure within the system. In one embodiment, the pressure sensor 23 can be used to prevent system pressure from moving above a predetermined ceiling. In another embodiment, the pressure sensor 23 can also be used to determine when the compression device 12 has completely deflated. Alternatively, the sensor 23 can be a position sensor 23 that is configured to monitor the position of the moveable wall 26 such that the sensor can sense when the moveable wall 26 is in the inflation position and/or the deflation position. In yet another alternative, both a pressure sensor and a position sensor can be provided. According to an additional implementation, the sensor 23 can be a combination pressure and temperature sensor 23. In a further alternative, instead of a sensor, the motor power signal can be used for the same purposes. Further, it is understood that any of these sensor embodiments can be used with any positive displacement pump implementation.

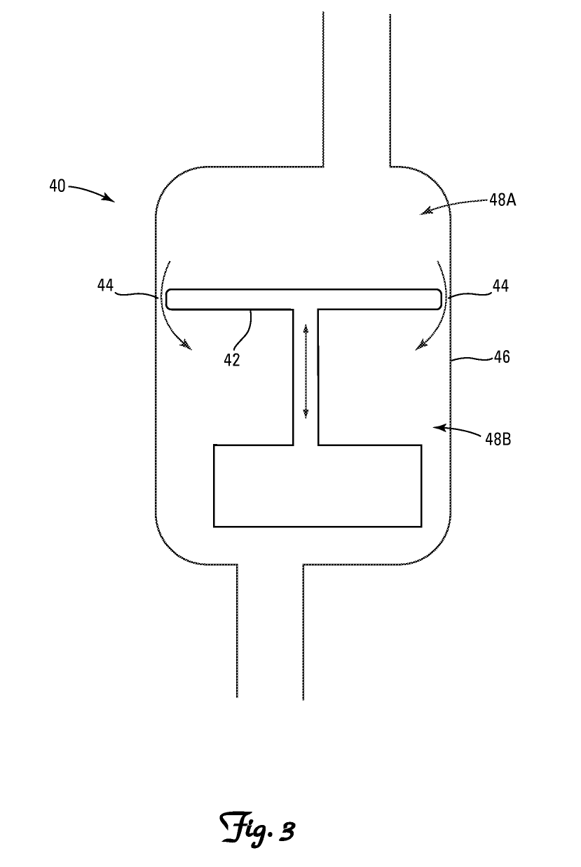

In an alternative implementation as shown in FIG. 3, the pump 40 is substantially similar to the positive displacement pump 20 described above and all of the discussion above applies equally to this pump 40. However, instead of fluid transfer holes as described above, the moveable wall 42 in this embodiment has fluid transfer gaps 44 defined between the ends of the moveable wall 26 and the inner walls of the pump body 46. As with the holes 34 described above, the fluid transfer gaps 44 are fluid transfer openings 44 that allow some predetermined amount of fluid to travel from one of the chambers 48A, 48B to the other through the gaps 44.

A further alternative embodiment of a positive displacement pump 130 is depicted in FIG. 10. The pump 130 has a pump body 132 and a moveable wall 134 that divides the body 132 into first and second chambers 136A, 136B and moves between a deflation position 134A and an inflation position 134B (depicted with broken lines). Instead of fluid transfer holes in the wall 134 (similar to the fluid transfer holes 34 in the wall 26 of FIG. 2), this pump 130 embodiment has a fluid transfer opening 138 that is a fluid transfer chamber 138 (also referred to herein as a “fluid transfer bypass chamber” or simply “bypass chamber”) defined in the wall of the body 132 that allows some amount of fluid within the body 132 to travel from one of the chambers 136A, 136B to the other through the bypass chamber 138. More specifically, in use, as the wall 134 moves into the inflation position 134B, the wall 134 is in close proximity to the wall of the pump body 132, thereby reducing, but not eliminating, the flow of fluid from one chamber 136A, 136B to the other. Alternatively, the wall 134 can substantially be in contact with the wall of the body 132 so long as no fluidic seal is established between the two walls such that some minimum amount of fluid is still allowed to travel from one of the chambers 136A, 136B to the other.

However, in this implementation, as the wall 134 moves into the deflation position 134A, the wall 134 moves into close proximity with the bypass chamber 138, thereby resulting in a larger gap between the wall 134 and the pump body 132 and thus allowing for fluid to flow from one chamber 136A, 136B to the other at a higher rate than when the wall is not in close proximity with the bypass chamber 138.

In use, the positioning of the fluid transfer chamber 138 results in a pump that has minimal leakage in the inflation position 134B, which results in slow deflation of the inflated compression device 12. In contrast, in the deflation position 134A, the bypass chamber 138 causes greater leakage at a faster rate (in comparison to the inflation position 134B), thereby resulting in faster flow of the fluid from the second chamber 136B to the first chamber 136A. This increased leakage or flow rate allows fluid that leaked from the first chamber 136A to the second chamber 136B when the wall 134 was in the inflation position 134B to flow back to the first chamber 136A, thereby allowing the pressure to be equalized between the two chambers 136A, 136B. This rapid flow rate quickly eliminates any excess fluid in either of the chambers 136A, 136B, thereby eliminating, or at least reducing, the risk of the moveable wall 134 moving back toward the inflation position 134B with a reduced amount of fluid positioned in the first chamber 136A such that the pressure in that chamber 136A cannot achieve the desired pressure as the wall 134 approaches the inflation position 134B. In one implementation, this fluid transfer chamber 138 is particularly effective when the patient's heart is beating at a fast rate (such as 160 bpm, for example) such that moveable wall 134 is moving quickly between the inflation 134B and deflation positions 134A. In such an embodiment, the ability to quickly balance the pressure in the two chambers 136A, 136B during the short time that the wall 134 is in proximity with the bypass chamber 138 can be important.

It is understood by those of skill in the art that, in certain embodiments, the need to balance the pressure between the two chambers 136A, 136B can involve flow in the other direction. That is, in certain embodiments, the compression device 12 may require force not only to inflate the device, but also to deflate the device 12 such that fluid leaks from the second chamber 136B to the first chamber 136A when the wall 134 is moved into the deflation position 134A.

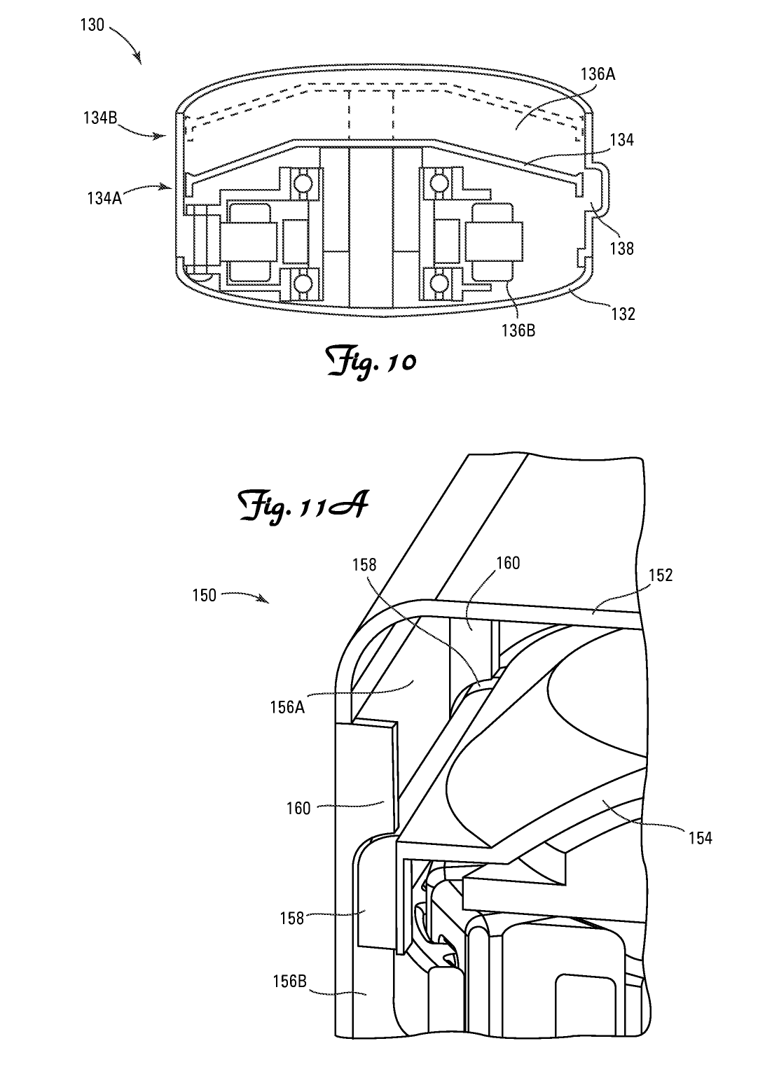

A further alternative implementation of a positive displacement pump 150 is depicted in FIGS. 11A and 11B, which are close-up views of the pump 150. While the entire pump 150 is not depicted, it is understood that according to certain embodiments, the pump 150 has a general configuration similar to FIGS. 2, 3, and 10. The pump 150 has a pump body 152 and a moveable wall 154 that divides the body 152 into first and second chambers 156A, 156B and moves between a deflation position (as shown in FIG. 11A) and an inflation position (as shown in FIG. 11B). As best shown in FIG. 11A, instead of fluid transfer holes or a fluid transfer chamber in the wall (similar to the chamber 138 in the wall of the body 132 as shown in FIG. 10), this pump 150 embodiment has one or more fluid transfer openings 158 that are fluid transfer slots 158 (also referred to herein as “bypass slots” 158) defined in the outer circumference of the moveable wall 154. These slots 158 allow some amount of fluid within the body 152 to travel from one of the chambers 156A, 156B to the other through the fluid transfer slots 158. In one embodiment as shown, the wall 154 has at least two slots 158. Alternatively, the wall 154 can have any number of predetermined slots 158 that allow the appropriate amount of fluid flow from one chamber 156A, 156B to the other. In this embodiment, the pump body 152 also has projections 160 defined in the inner wall of the body 152 that correspond to the slots 158. As shown in FIGS. 11A and 11B, the projections 160 are positioned on the body 152 such that they are positioned within the fluid transfer slots 158 when the moveable wall 154 is in the inflation position of FIG. 11B.

As such, in use, as the wall 154 moves into the inflation position (FIG. 11B), the projections 160 are positioned within the slots 158, thereby reducing the flow of fluid from one chamber 156A, 156B to the other. However, as the wall moves into the deflation position (FIG. 11A), the projections 160 are no longer positioned within the slots 158, thereby allowing for fluid to flow from one chamber 156A, 156B to the other through the slots 158 at a greater rate than when the projections 160 are positioned within the slots 158.

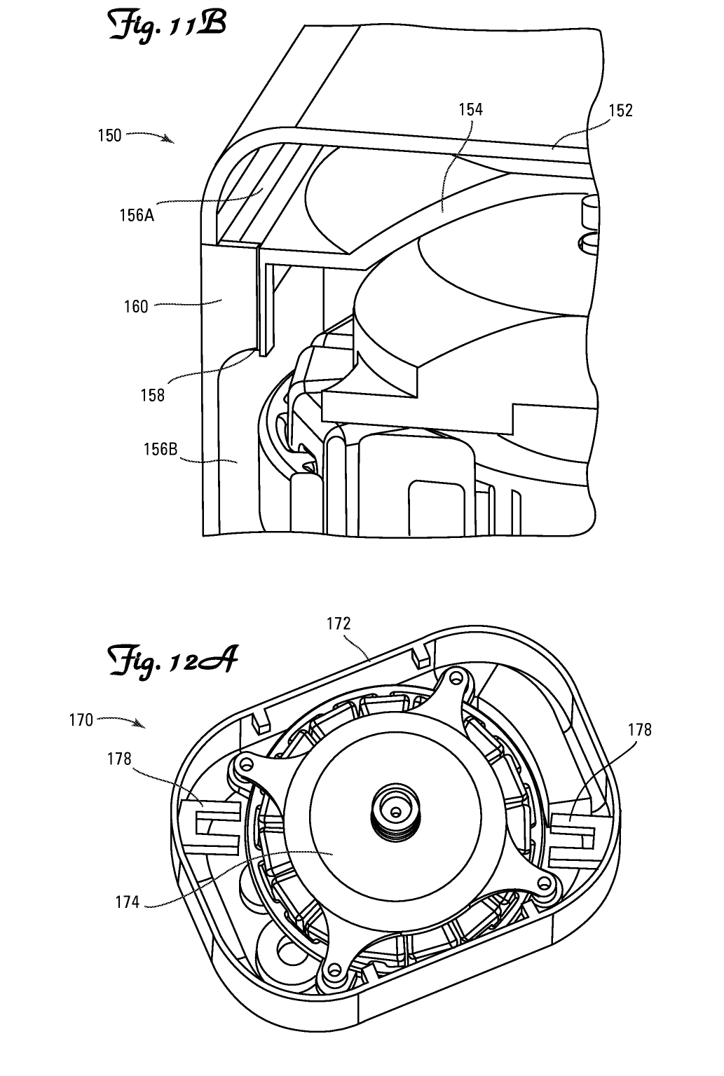

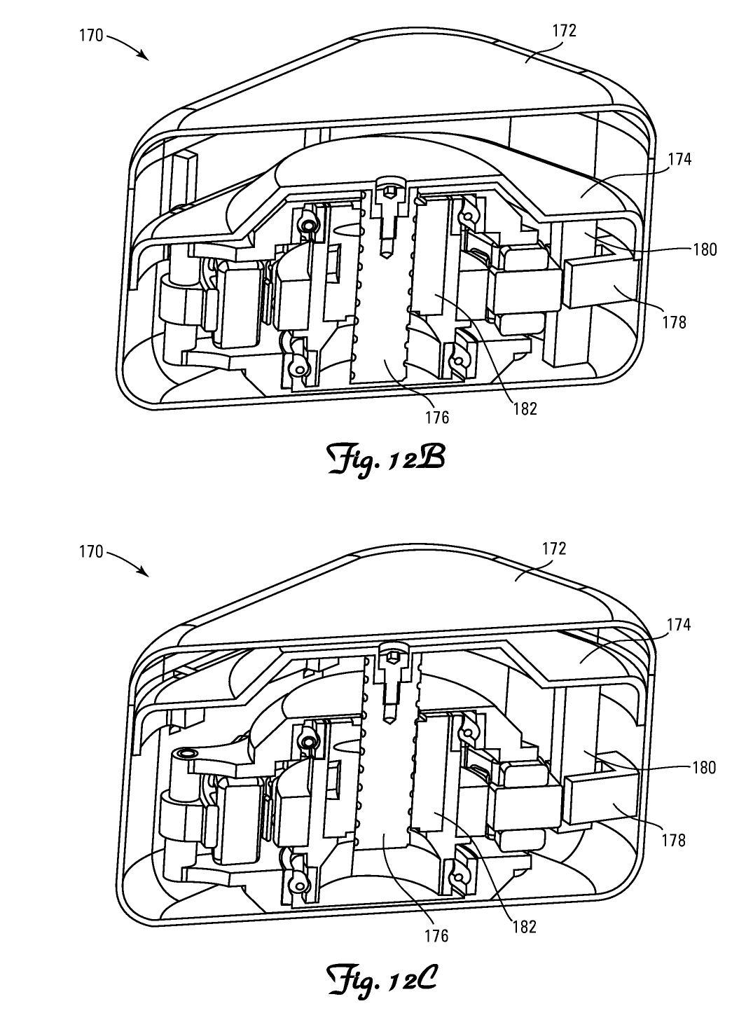

FIGS. 12A, 12B, and 12C depict another embodiment of a positive displacement pump 170 having a pump body 172 and a moveable wall 174. This pump embodiment is configured to prevent rotation of the moveable wall 154 in relation to the body 172. As best shown in FIGS. 12A and 12B, the pump 170 has a motor or actuation apparatus similar to the actuation components described in U.S. Pat. No. 7,306,558, which is hereby incorporated by reference in its entirety. More specifically, the pump 170 has a threaded shaft 176 that is fixedly coupled to the moveable wall 174. In this embodiment, a roller screw drive system 182 similar to the one described above and depicted in FIG. 4 is coupled to the motor, and the threaded shaft 176 is threadably coupled to the drive system 182. When the motor rotates the drive system 182 as described above, the shaft 176 is urged laterally in a direction that is parallel to the longitudinal axis of the shaft 176, which causes the wall 174 to move between the deflation position (in FIG. 12B) and the inflation position (in FIG. 12C). This actuation occurs because a portion of the roller screw drive system 182 rotates while the shaft 176 and the wall 174 do not. Thus, to ensure movement of the wall 174 between the deflation and inflation positions, it is important that the wall 174 and shaft 176 are refrained from rotating.

Alternatively, a ball screw drive system could be used in this embodiment as well. In a further alternative, any known motor for use in medical devices that can actuate the wall 174 to move laterally can be used in the current implementation.

In certain positive displacement pump embodiments as discussed above (such as, for example, the pump 20 depicted in FIG. 2), the moveable wall is restrained from rotating by a non-rigid coupling component (such as the component 32 in FIG. 2), which couples the moveable wall to the wall of the pump body (in addition to maintaining a fluidic seal between the two chambers of the pump). However, in embodiments such as the pump 170 in FIGS. 12A, 12B, and 12C that has no such non-rigid coupling component, another mechanism or structure must be provided to restrain the moveable wall 174. Thus, the pump 170, as best shown in FIG. 12A, has two magnetic slots 178 protruding from the interior wall of the pump body 172. As best shown in FIGS. 12B and 12C, the moveable wall 174 has at least one piston 180 that is coupled to and extends from the wall 174 as shown, and each such piston 180 is configured to be positioned through one of the magnetic slots 178. The piston 180 interacts magnetically with the slot 178 such that the slot 178 retains the piston 180 in its position through the slot 178 and thus restrains the moveable wall 174 from rotating. In one implementation, the magnetic communication between each slot 178 and piston 180 applies magnetic forces to each piston 180 that help to prevent the piston 180 from coming into physical contact with the slot 178. Despite the rotational restraint, the piston 180 is allowed to move up and down through the slot 178 such that the moveable wall 174 can move between the deflation position in FIG. 12B and the inflation position in FIG. 12C.

In the specific embodiment depicted in FIGS. 12A-12C, the positive displacement pump 170 has two magnetic slots 178 as best shown in FIG. 12A and two pistons 180, one for each slot 178 (only one piston 180 is depicted in FIGS. 12B and 12C). Alternatively, the pump 170 can have one slot 178 (and one corresponding piston 180). In a further alternative, the pump 170 can have three or more slots 178 and three or more corresponding pistons 180. The slot(s) 178 can also be any other known structural feature that can retain the piston 180 and thus the wall 174 from rotating. Further, the slot(s) 178 can also be non-magnetic.

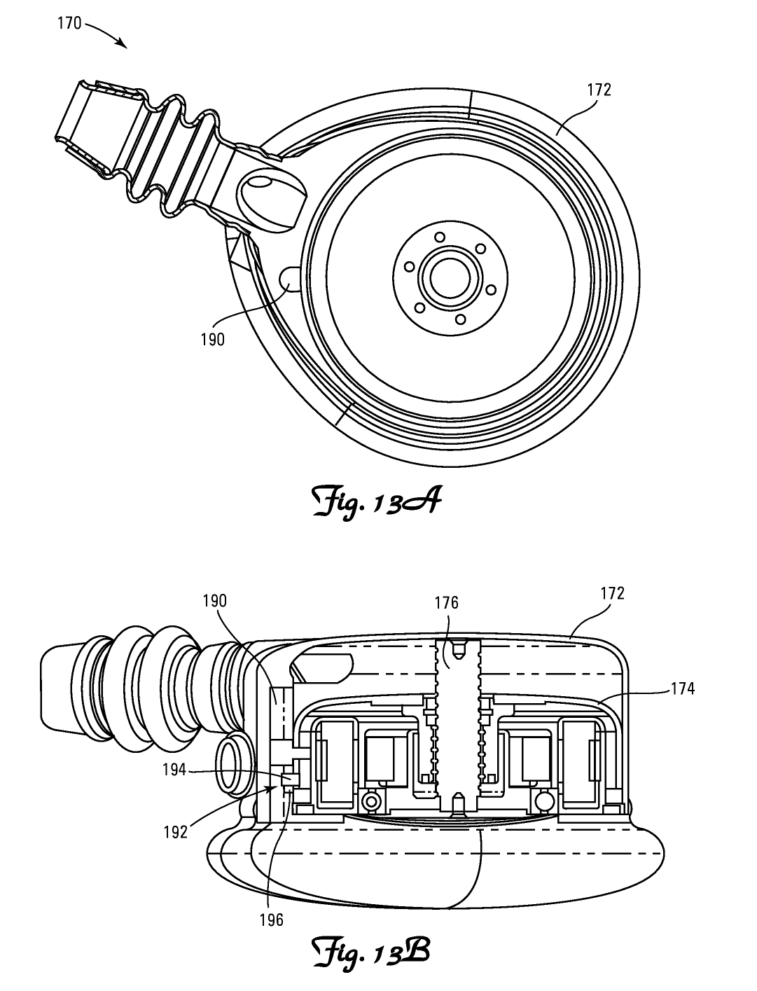

Another implementation is shown in FIGS. 13A and 13B in which the pump 170 has no non-rigid coupling component and instead has a mechanical, non-magnetic, slidable coupling that allows for movement of the moveable wall 174 between the deflation and inflation positions while preventing the wall 174 from rotating. More specifically, in this embodiment, the pump 170 has a slot 190 defined in a portion of the interior wall of the body 172 (as best shown in the cross-sectional, cutaway top view of FIG. 13A in combination with the cross-sectional, cutaway side view of FIG. 13B) and extends along the wall such that the slot 190 is parallel with the threaded shaft 176 as shown. The moveable wall 174 of the pump 170 has a protrusion 192 that is configured to be mateable to and fit within the slot 190 in the body 172. In one embodiment, the protrusion 192 is made up of a rod, bolt, or pin 194 extending axially into the slot 190 with a bearing 196 disposed around the pin 194. In accordance with one implementation, the bearing 196 is a rotatable bearing 196 such that the bearing 196 can rotate within the slot 190 as the moveable wall 174 moves between its deflation and inflation positions. The protrusion 192 interacts mechanically with the slot 190 such that the protrusion 192 is retained within the slot 190 while the moveable wall 174 moves between the deflation and inflation positions, thereby preventing the wall 174 from rotating. In the specific embodiment depicted in FIGS. 13A and 13B, the pump 170 has one slot 190. Alternatively, the pump 170 can have two or more slots 190 with a corresponding number of protrusions 192.

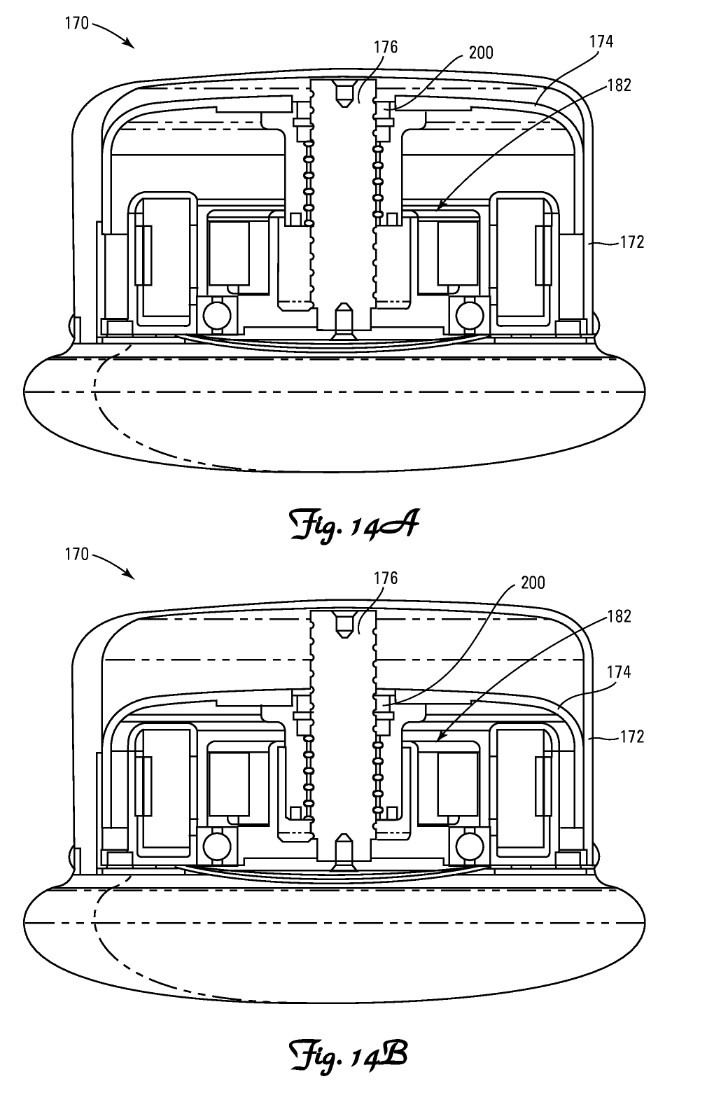

In an alternative implementation shown in FIGS. 14A and 14B, the threaded shaft 176 is configured such that it cannot move laterally but is allowed to rotate, and the moveable wall 174 is configured to move laterally along the shaft 176 via a nut 200 that is threadably engaged with the threaded shaft 176. The nut 200 is coupled to the moveable wall 174 such that neither the nut 200 nor the wall 174 can rotate. Thus, rotation of the shaft 176 causes the nut 200 to move laterally, thereby causing the moveable wall 174 to move laterally between the inflation position in FIG. 14A and the deflation position in FIG. 14B. The drive system 182 is fixedly coupled to the device body 172. In use, the shaft 176 is rotated by the drive system 182, thereby causing the non-rotatable nut 200 to move laterally, thereby causing the moveable wall 174 to move laterally, thereby urging the wall 174 between the deflated position (FIG. 14B) and inflated position (FIG. 14A). The drive system 182 can have any known motor for use in medical devices that can actuate the wall 174 to move laterally.

In other embodiments, the pumps contemplated herein are gear pumps. For example, according to one embodiment, FIG. 5 depicts another pump 60 for use with systems such as the heart assist system 10 discussed above. This pump 60 is an internal gear pump 60 that is also known as a gerotor 60. The gerotor 60 is a positive displacement pumping device that has an inner rotor 62 and an outer rotor 64. As shown in FIG. 5, the outer rotor 64 has one more tooth than the inner rotor 62 and has its axis positioned at a fixed eccentricity in relation to the axis of the inner rotor 62.

According to one embodiment, the internal gear pump 60 is self-priming and can run dry for short periods. Further, this pump 60 is bi-rotational, meaning that the rotors 62, 64 can rotate in either direction. As such, the rotors 62, 64 can be rotated in one direction to inflate the compression device 12 and in the other direction to deflate it. In accordance with one implementation, this pump 60 and other internal gear pumps have only two moving parts. As such, they are generally reliable, simple to operate, and easy to maintain in comparison to pumps with more moving parts.

In use, fluid enters the suction port 66 between the outer rotor 64 and the inner rotor 62 teeth. As shown in FIG. 5, the arrows indicate the direction of the fluid. The rotation of the rotors 62, 64 urges the liquid to travel through the pump 60 between the teeth of the rotors 62, 64.

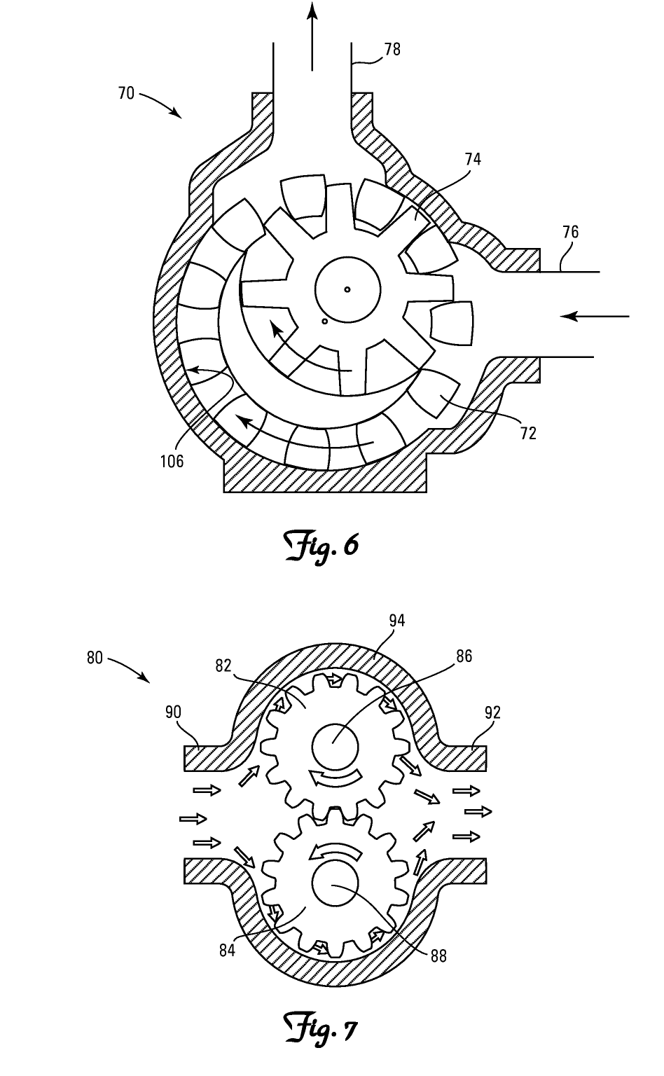

FIG. 6 depicts an alternative embodiment of a pump 70. This internal gear pump 70 is an alternative version of a gerotor 70. Like the pump in FIG. 5, this pump 70 has an outer rotor 72 and an inner rotor 74 (also referred to as an “idler”). The idler 74 has its axis positioned at a fixed eccentricity in relation to the axis of the outer rotor 72 such that the teeth of the idler 74 and the outer rotor 72 mesh to form a seal between the intake port 76 and the discharge port 78, which forces the liquid out of the discharge port 78. It is understood that in certain embodiments, the seal formed between the teeth of the idler 74 and the outer rotor 72 is not a complete seal but rather an effective seal, thereby allowing for some flow as discussed below. In addition, the intermeshing teeth of the idler 74 and rotor 72 form effectively, but not completely, fluidly sealed pockets for the fluid, which assures volume control.

Both of the pump embodiments 60, 70 discussed above are configured to allow fluid to leak or flow back from the high pressure side of the rotors to the lower pressure side, thereby allowing the compression device 12 to deflate in the case of an unexpected pump stoppage similar to that described above. That is, each pump 60, 70 has a fluid transfer opening that allows flow of fluid similar to the various fluid transfer openings discussed above. These flow-back configurations will be discussed in further detail below.

In one implementation, an advantage of a gear pump such as the gear pumps described herein is that it can be smaller in comparison to some other types of pumps because the displacement volume is used multiple times with each revolution of the rotors. As such, a gear pump can help to optimize the amount of space necessary for the overall heart assist system such as the system 10 described above.

FIG. 7 depicts an alternative embodiment of a gear pump 80. In contrast to the pumps 60, 70 depicted in FIGS. 5 and 6 and discussed above (which were internal gear pumps), this pump 80 is an external gear pump 80. Like the internal gear pumps, this external gear pump 80 has two gears 82, 84 that mesh together at a single area or point of contact to produce flow. However, the external gear pump 80 has two gears 82, 84 that rotate in opposite directions. According to one embodiment, one of the two gears is operably coupled to a motor (not shown) such that the motor drives that gear, and that gear in turn drives the other gear. In accordance with one implementation, each of the gears 82, 84 is supported by a shaft 86, 88 with bearings (not shown) on both sides of the gear.

In use, as the two gears 82, 84 rotate and the teeth of the gears 82, 84 exit from the area where the teeth mesh with each other, the movement of the teeth creates expanding volume inside the intake port 90. This causes fluid to flow into the intake port 90. The gear teeth draw the fluid toward the inner walls of the pump 80 and thus cause the fluid to be pulled around the outside of the gears 82, 84 between the teeth and the inner wall of the pump body 94. The rotation of the gears 82, 84 and the meshing of the teeth urge the fluid out of the pump through the discharge port 92.

It is understood that the gear pump embodiments described herein each have a motor that actuates the rotary motion of the pumps. It is further understood that each of the various gear pump embodiments disclosed herein can operate in both directions, thereby allowing the pump to both inflate and deflate the compression device 12. Further, it is understood that the positive displacement nature of these gear pumps results in a known number of gear rotations displacing a known amount of liquid (given some leakage).

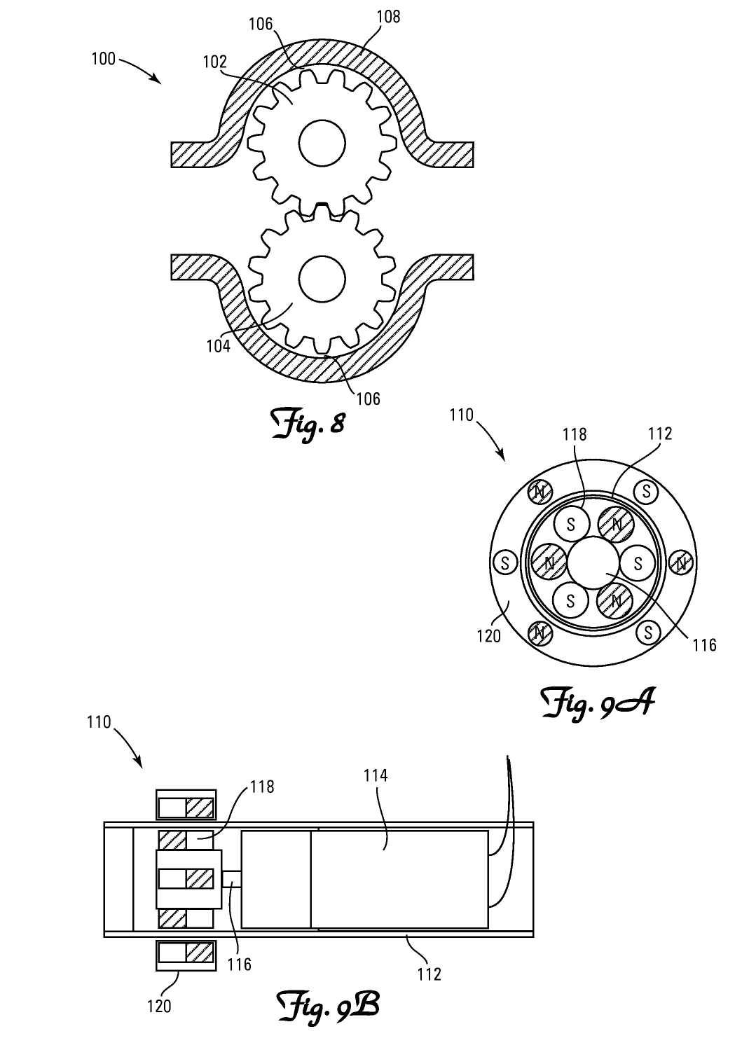

FIG. 8 depicts a particular embodiment of an external gear pump 100 that has been configured to allow for flow of fluid from the high pressure side of the pump to the low pressure side. That is, the pump 100 has been made to allow for fluid back flow, or, in other words, to be “deliberately leaky.” Like the embodiments discussed above, this allowance of “back flow” addresses the risk associated with a prolonged stoppage of the pump 100 (relative to the cardiac cycle) as a result of the pump 100 getting stuck or a complete power failure or any other issue that leaves the compression device 12 in the inflated state. In this embodiment, the device 100 is configured to allow “back flow” by creating fluid transfer openings or fluid transfer gaps 106 of predetermined size between the teeth of the two gears 102, 104 and the inner wall of the pump body 108. As discussed above, the size of the fluid transfer gaps 106 can be predetermined to create a predetermined amount of back flow of the fluid from the high pressure to the low pressure side, thereby resulting in a predetermined rate of deflation of the compression device 12.

Similarly, as mentioned above with respect to the embodiments depicted in FIGS. 5 and 6, both of the pump embodiments 60, 70 are also configured to allow fluid to leak or flow back from the high pressure side of the rotors to the lower pressure side. That is, like the external pump 100 of FIG. 8 and discussed above, each of the pumps 60, 70 can be configured in certain implementations to allow “back flow” via fluid transfer openings or gaps of predetermined size. For the pump 60 in FIG. 5, the fluid transfer gap 106 would be between the inner rotor 62 and an outer rotor 64. With respect to pump 70 in FIG. 6, the fluid transfer gap 106 would be between the outer rotor 72 and the inner wall of the pump 70. While the gaps 106 as shown in FIGS. 5 and 6 are relatively small, it is understood that the gap 106 in each embodiment can be any appropriate size to allow for the appropriate amount of “back flow” as described with respect to other embodiments above. That is, as discussed above, in each case, the size of the fluid transfer gaps can be predetermined to create a predetermined amount of back flow of the fluid from the high pressure to the low pressure side, thereby resulting in a predetermined rate of deflation of the compression device 12.

For gear pumps, in one embodiment, the electrical power draw and speed signals from the pump motor (not shown) can be used to determine pressure within the compression device 12. This could allow for control against pressure limits and to determine when all fluid has been removed from the compression device 12. Alternatively, a pressure sensor (not shown) can be positioned within the liquid of any of the gear pump embodiments to sense pressure and thereby be used to prevent predetermined pressure limits being exceeded and further to determine when complete device 12 deflation has been achieved.

In one embodiment, the fluid used with the gear pump embodiments is silicone oil. Alternatively, the fluid is saline. In another alternative, the fluid can be any of the fluids discussed above with respect to the displacement pump embodiments. In a further embodiment, the fluid is any biocompatible and sterilizable fluid that can be used in medical devices implanted inside the human body.

According to one implementation, the motor (not shown) coupled to the gears in any of the gear pump embodiments is positioned in the fluid such that the seal between the shaft and the pump does not need to be hermetic. Similarly, the motor in any of the positive displacement embodiments can be positioned in the fluid.

Alternatively, as shown in FIGS. 9A and 9B, a motor assembly can be provided that actuates a gear pump without direct contact between the motor and the fluid. In this embodiment, the motor assembly 110 (as best shown in FIG. 9B) has a body 112 that is fluidically sealed so that the components inside the body 112 are not in contact with the fluid and the fluid cannot access any interior portion of the body 112 such that the motor 114 disposed in the body 112 has no contact with the fluid. The motor 114 actuates a pump (not shown) in the following fashion. The motor 114 is operably coupled to a shaft 116 that is operably coupled at its other end to a set of rotatable internal magnets 118, as best shown in FIG. 9A. Further, the assembly 110 also has a set of rotatable external magnets 120. In use, the motor 114 actuates the rotation of the internal magnets 118 via the shaft 116. The rotation of the internal magnets 118 causes the rotation of the external magnets 120 as a result of the magnetic forces interacting between the two sets of magnets 118, 120. Thus, the actuation of the motor 114 inside the fluidically sealed body 112 causes the rotation of the external magnets 120, thereby actuating the pump (not shown), which is mechanically coupled to the motor assembly 110.

In one embodiment, one advantage of this magnet-based motor assembly is that it limits the amount of torque that can be transmitted and thereby limits the pressure the pump (not shown) can apply.

Alternatively, the pump gear (not shown) can also serve as the rotor of the motor and stator coils can be positioned externally around the rotor. In this arrangement, the gear is a part of the electric motor rather than a separate element.

It is understood that this motor assembly 110 depicted in FIGS. 9A and 9B can also be used with any of the positive displacement pump embodiments disclosed or contemplated herein.

While multiple embodiments are disclosed, still other embodiments will become apparent to those skilled in the art from the detailed description, which shows and describes illustrative embodiments of the invention. As will be realized, the various embodiments are capable of modifications in various obvious aspects, all without departing from the spirit and scope of the various inventions. Accordingly, the drawings and detailed description are to be regarded as illustrative in nature and not restrictive.

What is claimed is:

1. An pump for a medical device, the pump comprising:
(a) a body defining an interior;
(b) a displacement component disposed within the interior;
(c) a first chamber defined by a distal portion of the body and a distal side of the displacement component;
(d) a conduit in fluid communication with the first chamber, the conduit being in fluid communication with the medical device;
(e) a second chamber defined by a proximal portion of the body and a proximal side of the displacement component; and
(f) at least one fluid transfer opening defined between the first chamber and the second chamber.

2. The pump of claim 1, wherein the medical device is an inflatable compression device.

3. The pump of claim 2, wherein the at least one fluid transfer opening is sized and shaped to allow the compression device to deflate within a time period ranging from about 10 seconds to about 30 seconds.

4. The pump of claim 2, wherein the at least one fluid transfer opening is sized and shaped to allow a maximum flow rate through the opening of about 2 cc per second.

5. The pump of claim 1, wherein the displacement component comprises a displacement wall.

6. The pump of claim 5, wherein the at least one fluid transfer opening comprises an opening defined in the displacement wall.

7. The pump of claim 6, further comprising a non-rigid coupling component operably coupled to the displacement wall and an interior wall of the body.

8. The pump of claim 5, wherein the at least one fluid transfer opening comprises a gap between the displacement wall and an interior wall of the body.

9. The pump of claim 5, wherein the at least one fluid transfer opening comprises a bypass chamber defined in the body.

10. The pump of claim 9, wherein the displacement wall is positioned adjacent to the bypass chamber when the displacement wall is in a deflation position.

11. The pump of claim 5, wherein the at least one fluid transfer opening comprises at least one slot defined in the displacement wall, wherein the implantable pump further comprises at least one projection shaped to fit within the slot.

12. The pump of claim 11, wherein the at least one projection is disposed within the at least one slot when the displacement wall is in an inflation position.

13. The pump of claim 1, wherein the displacement component comprises an at least one rotor.

14. The pump of claim 1, wherein the displacement component comprises a first rotor and a second rotor.

15. An pump for a medical device, the pump comprising:
(a) a body defining an interior;
(b) a displacement wall disposed within the interior;
(c) a first chamber defined by a distal portion of the body and a distal side of the displacement wall;
(d) a conduit in fluid communication with the first chamber, the conduit being in fluid communication with the medical device;
(e) a second chamber defined by a proximal portion of the body and a proximal side of the displacement wall;
(f) a compliance chamber in fluid communication with the second chamber; and
(g) at least one fluid transfer opening defined between the first chamber and the second chamber.

16. The pump of claim 15, wherein the at least one fluid transfer opening comprises an opening defined in the displacement wall.

17. The pump of claim 15, wherein the at least one fluid transfer opening comprises a gap between the displacement wall and an interior wall of the body.

18. An gear pump for a medical device, the pump comprising:
(a) a body defining an interior;
(b) at least one rotor disposed within the interior;
(c) a first chamber defined by a distal portion of the body and a distal portion of the at least one rotor;
(d) a conduit in fluid communication with the first chamber, the conduit being in fluid communication with the medical device;
(e) a second chamber defined by a proximal portion of the body and a proximal portion of the at least one rotor; and
(f) at least one fluid transfer opening defined between the first chamber and the second chamber.

19. The gear pump of claim 18, wherein the at least one rotor comprises a first rotor and a second rotor.

20. The gear pump of claim 18, wherein the at least one fluid transfer opening comprises a gap between the at least one rotor and an interior wall of the body.
